(19) United States
(12) Patent Application Publication
(10) Pub. No.: US 2023/0285612 A1
(43) Pub. Date: Sep. 14, 2023

(54) UV LIGHT HOUSING

(71) Applicant: Mohammad Nasser, Phoenix, AZ (US)

(72) Inventor: Mohammad Nasser, Phoenix, AZ (US)

(21) Appl. No.: 18/119,334

(22) Filed: Mar. 9, 2023

Related U.S. Application Data
(60) Provisional application No. 63/318,116, filed on Mar. 9, 2022.

(51) Int. Cl. A61L 2/10

(52) U.S. Cl. CPC A61L 2/10; A61L 2202/11; A61L 2202/121

(57) ABSTRACT

A UV light housing comprised of a power source, a housing shaped to conform to a contact surface, and one or more UV light sources. The power source may be a battery, a rechargeable battery, kinetic energy, solar, or it may connect to a power grid. The UV light sources are placed internally, externally, or a combination of internal and external locations of the housing. The housing can be of any form including a door handle, a pull handle, a push plate, a pen, a drinking fountain guard, and a condiment pump handle. The UV light source(s) may be configured to be always on, to turn on once the contact surface has been used, or it may turn on with a sensor.

### CROSS REFERENCE TO RELATE APPLICATION

This application claims benefit from currently pending U.S. Provisional Application No. 63/318,116 titled “UV LIGHT HOUSING” and having a filing date of Mar. 9, 2022, all of which is incorporated by reference herein.

### TECHNICAL FIELD

The present specification relates generally to housings for high contact areas, such as door handles, and more particularly to such devices having self-sterilizing properties.

### BACKGROUND

With epidemics and pandemics have come a heightened awareness for the need of limiting a user's exposure to live microorganisms such as viruses, bacteria, fungi, and other microbes. However, the way to limit this exposure in public areas, and more specifically on public contact surfaces, has been difficult to navigate.

Many public contact surfaces are made with highly durable materials such as stainless steel or hard plastics. These materials are great for durability, but do not inhibit the growth or spread of microorganisms that are deposited from user's hands onto the surface. This facilitates the contamination of user's hands as they pick up microorganisms from the shared use of the public contact surface, such as door handles, gas pumps, drinking fountains, pens, and the like. Common places for the public contact surfaces are schools, hotels, airports, restaurants, and other public buildings. The spread of live microorganisms may be particularly problematic in hospitals, food processing facilities, or laboratories, where the consequences of the spreading of live microorganisms may be most severe. Several attempts to mitigate the propagation of live microorganisms have been attempted such as disinfectant sprays or pumps, however these rely on a user being diligent in maintaining sanitary conditions with every contact instance. These also rely on a user being thorough in their cleaning of contact surfaces, some of which are shaped so that thorough cleaning is difficult or impossible. These approaches also require the disinfectant spray or pump to be filled regularly, the neglect of which leaves unchecked propagation of microorganisms.

### SUMMARY

Accordingly, the present UV light housing has been made in view of the above-mentioned disadvantages occurring in the prior art. One advantage of the UV light housing is to allow for consistent, thorough method of killing or inactivating microorganisms on public contact surfaces regardless of a user's sanitary habits.

In one embodiment the UV light housing comprises a power source, a housing shaped to be a contact surface, and a plurality of UV light sources. The housing has a contact side, and an inner side. The UV light sources may be positioned about the housing to illuminate the contact surface with UV light rays sufficient to kill or inactivate microorganisms on the contact surface.

A UV light system for a contact item, the system comprising a power source and a transparent housing defining a system contact surface having a contact side and an inner side. A plurality of UV light sources positioned on the inner side of the housing, wherein the plurality of UV sources can be configured to illuminate the contact surface with UV light rays sufficient to provide at least a 2-log reduction in the number of microorganisms on the contact surface. The power source can be a rechargeable battery. The power source can be kinetic energy from the door opening and closing. The lights can be connected to a power grid. The power source can be at least one photovoltaic panel. The contact surface can be found on a door handle, a door push plate, a pen, a drinking fountain guard, a drinking fountain push bar, or a condiment pump handle.

The housing conforms to an existing contact surface of a contact item such that the system contact surface replaces the existing contact surface. A UV light system for a contact item, the system comprising a power source. A transparent housing defining a system contact surface having a contact side and an inner side, wherein the housing can be comprised of a first section and a second section detachably coupled to the first section and wherein the housing conforms to an existing contact surface of a contact item such that the system contact surface replaces the existing contact surface. A plurality of UV light sources positioned on the inner side of the housing, wherein the plurality of UV sources can be configured to illuminate the contact surface with UV light rays sufficient to provide at least a 2-log reduction in the number of microorganisms on the contact surface. The first section and second section can be mirror images of each other.

A method of providing a disinfected contact surface of a contact item, the method comprising the acts of forming a housing to conform to an existing contact surface, wherein the housing is substantially transparent and defines a system contact surface having a contact side and an inner side, wherein the housing is comprised of a first section and a second section detachably coupled to the first section such that the system contact surface replaces the existing contact surface, wherein the housing further comprises a plurality of UV light sources positioned on the inner side of the housing and are configured to illuminate the contact surface with UV light rays.

Connecting the first section and the section to replace the existing contact surface. Operating the UV light sources to illuminate the system contact surface sufficient to provide at least a 2-log reduction in the number of microorganisms on the system contact surface. The existing contact surface is replaced by the system contact surface by removing the existing contact surface and inserting the system contact surface. The existing contact surface can be replaced by the system contact by attaching the first section to the second section about the existing contact surface.

Aspects and applications of the invention presented here are described below in the drawings and detailed description of the invention. Unless specifically noted, it is intended that the words and phrases in the specification and the claims be given their plain, ordinary, and accustomed meaning to those of ordinary skill in the applicable arts. The inventors are fully aware that they can be their own lexicographers if desired. The inventors expressly elect, as their own lexicographers, to use only the plain and ordinary meaning of terms in the specification and claims unless they clearly state otherwise and then further, expressly set forth the. Absent such clear statements of intent to apply a “special” definition, it is the inventor's intent and desire that the simple, plain, and ordinary meaning to the terms be applied to the interpretation of the specification and claims.

The inventors are also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventors are fully informed of the standards and application of the special provisions of 35 U.S.C. § 112 (f). Thus, the use of the words “function,” “means” or “step” in the Detailed Description or Description of the Drawings or claims is not intended to somehow indicate a desire to invoke the special provisions of 35 U.S.C. § 112 (f), to define the invention. To the contrary, if the provisions of 35 U.S.C. § 112 (f) are sought to be invoked to define the inventions, the claims will specifically and expressly state the exact phrases “means for” or “step for” and will also recite the word “function” (i.e., will state “means for performing the function of . . . , without also reciting in such phrases any structure, material or act in support of the function. Thus, even when the claims recite a “means for performing the function of . . . ,” if the claims also recite any structure, material or acts in support of that means or step, or that perform the recited function, then it is the clear intention of the inventors not to invoke the provisions of 35 U.S.C. § 112 (f). Moreover, even if the provisions of 35 U.S.C. § 112 (f) are invoked to define the claimed inventions, it is intended that the inventions not be limited only to the specific structure, material or acts that are described in the preferred embodiments, but in addition, include any and all structures, materials or acts that perform the claimed function as described in alternative embodiments or forms of the invention, or that are well known present or later-developed, equivalent structures, material or acts for performing the claimed function.

Additional features and advantages of the present specification will become apparent to those skilled in the art upon consideration of the following detailed description of the illustrative embodiment exemplifying the best mode of carrying out the invention as presently perceived.

### BRIEF DESCRIPTION OF THE DRAWINGS

These and other features, aspects, and advantages of the present specification will become better understood with regard to the following description, appended claims, and accompanying drawings where:

FIG. 1 is a front view of a door handle shaped housing in accordance to one, or more embodiments;

FIG. 2 is a iso-metric view of a pull shaped housing in accordance to one, or more embodiments;

FIG. 3 is a front view of a push plate housing in accordance to one, or more embodiments;

FIG. 4 is an isometric view of a gas pump handle housing in accordance to one, or more embodiments;

FIG. 5 is an isometric view of a pen housing in accordance to one, or more embodiments;

FIG. 6 is an isometric view of a drinking fountain guard and push bar in accordance to one, or more embodiments; and

FIG. 7 is an isometric view of condiment pump in accordance to one, or more embodiments.

### DETAILED DESCRIPTION

This disclosure relates to a UV light housing. The UV light housing may be used as a public contact surface, such as, for example, a door handle, a pull handle, a push plate, a gas pump handle, a pen body, a drinking fountain guard, and the like. This UV light housing allows for many users to touch public contact surfaces without propagating the spread of microorganisms, and the diseases they cause.

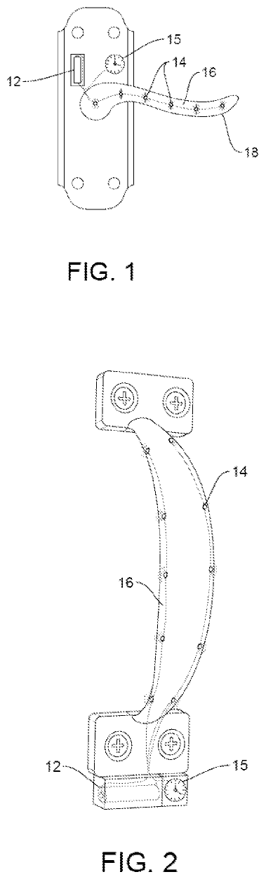

Referring initially to FIG. 1-2, differing embodiments of a UV light housing 10 is shown. The UV light housing comprises a power source 12, a UV light source 14, and a contact surface housing 16. The power source 12 may be a battery, a rechargeable battery, kinetic energy, photovoltaic array, power from a power grid, or the like. The power source 12 may be internal, such as with a battery, or it may be external, such as with the power grid hooked to a structure. The power source 12 may be made of multiple parts that can be such as, for example, internal, external, or combination of internal and external parts such as batteries, solar panels, voltage regulator, or the like. The UV light source 14 is electrically connected to the power source 12. In one or more embodiments the UV light source is a plurality of UV sources. In other embodiments the UV light source may be a single UV light source or a UV light source that is dispersed through a material to emit over a desired surface area.

In a particular embodiment the contact surface housing 16 is configured to be a door handle 18, and the UV light source 14 is a plurality of light sources placed along the surface of the handle 18 that is frequently contacted through normal use. The light source 14 could be placed along the interior and/or exterior surface of the contact surface housing 16, or a combination of internal and external placement. The door handle 18 can be made from any durable material such as metal, plastic, glass, fiberglass, and the like.

In one or more embodiments the UV light sources 14 may be configured to be always on, or they may be configured to turn on once the public contact surface 16 has been used, such as, for example, by turning of the door handle 18, or the opening and/or closing of a door. The UV light sources 14 may be on a sensor such as, for example a proximity sensor, hall effect sensor, motion sensor, infrared sensor or the like. The sensor may be configured to turn on the lights as a user approaches or leaves the area. The UV light sources 14 may be on a timer 15 to ensure the microorganisms have been exposed to a sufficient amount of light to kill or inactivate them. The timer 15 may be set to reduce the quantity of microbes on the contact surface 16 by a desired log reduction. For example, the timer may be calibrated to provide a 6-log reduction, or, in other words, be 99.9999% effective. In certain embodiments a microbe detection sensor can detect when the microbes are reduced and turn off the lights accordingly.

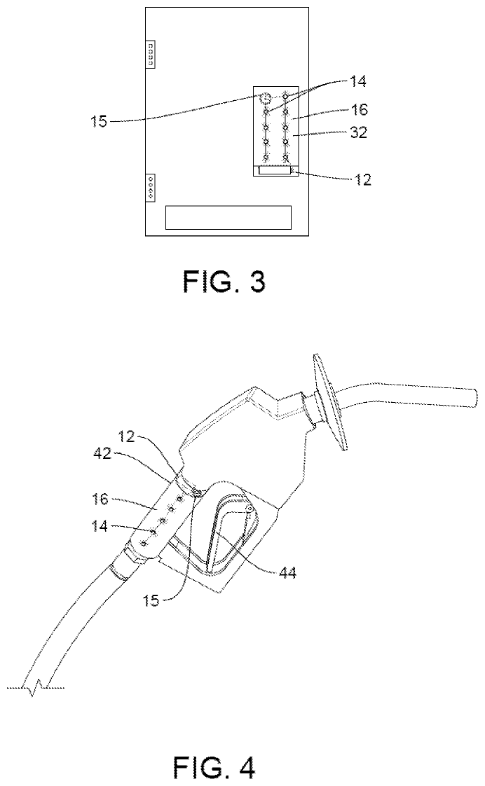

Referring now to FIG. 3 the front view of an embodiment of the UV light housing is shown as a push plate 32. A push plate 32 may be a generally flat and shallow plate, typically secured onto a door to protect the door surface for high contact areas on the door, such as in the middle of the non-hinge side of the door, where the door is pushed to open. Here the push plate 32 is shown to contain a plurality of UV light sources 14, but it may have a single source, more, or fewer light sources. The light sources 14 may be configured to expose the push plate 32, the surrounding areas of a door, and a door frame to UV light rays.

In one or more embodiments the UV light sources 14 may be configured to be always on, or they may be configured to turn on once the public contact surface 16 has been used, such as, for example, pressing the push plate 38, or the opening and/or closing of a door. The UV light sources 14 may be on a sensor. The sensor may be configured to turn on the lights as a user approaches or leaves the area. The UV light sources 14 may be on a timer to ensure the microorganisms have been exposed to a sufficient amount of light to kill or inactivate them or reduce them by a desired log reduction.

Referring now to FIG. 4 the isometric view of an embodiment of the UV light housing is shown as a gas pump handle. The power source 12 may be a battery, a rechargeable battery, kinetic energy, photovoltaic array, power from the power grid or from the gas pump, or the like. The power source 12 may be internal, such as with a battery, or it may be external, such as with the power grid. The power source 12 may be made of multiple parts that may be internal, external, or combination of internal and external. In this embodiment the UV light source 14 is a plurality of light sources placed along the interior or exterior of the handle 18. However, the light source 14 could be placed along the exterior surface of the contact surface housing 16, or a combination of internal and external placement. The UV light sources 14 could be placed, for example, on the handle configured to expose a top and a bottom of a lever 44 to the UV light rays. The UV light sources could extend the entire length of the pump handle 42, and not be constrained to the high contact areas of the pump handle 42. The pump handle 42 can be made from any durable material such as metal, plastic, glass, fiberglass, and the like.

In one or more embodiments the UV light sources 14 may be configured to be always on, or they may be configured to turn on once the public contact surface 16 has been used, such as, for example, when the gas pump handle is returned to the handle cradle. The UV light sources 14 may be on a timer to ensure the microorganisms have been exposed to a sufficient amount of light to kill or inactivate them or reduce them by a desired log reduction.

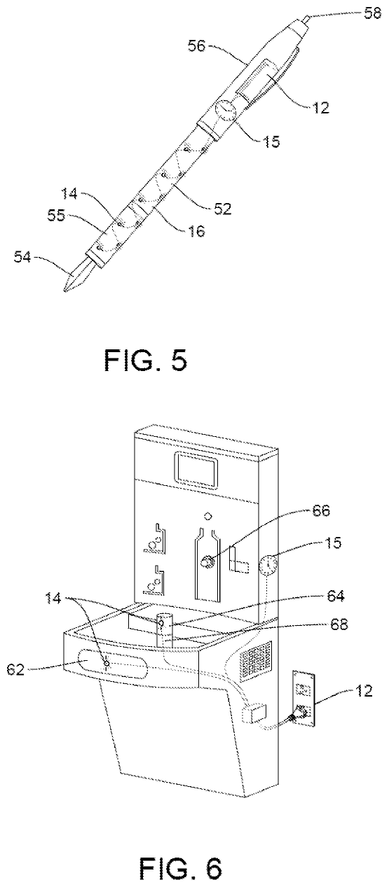

Referring now to FIG. 5 the isometric view of an embodiment of the UV light housing is shown as a pen 52. The power source 12 may be a battery, a rechargeable battery, kinetic energy, photovoltaic array, or the like. In this embodiment the UV light source 14 is a plurality of light sources placed along the interior of the pen grip 55. The light sources 14 could be extended along the pen barrel 56. The light source 14 could be placed along the exterior surface of the pen 52, or a combination of internal and external placement. The pen 52 can be made from many materials such as metal, plastic, glass, fiberglass, and the like.

In one or more embodiments the UV light sources 14 may be configured to be always on, or they may be configured to turn on once the public contact surface 16 has been used, such as, for example, when the end cap 58 is depressed. The UV light sources 14 may be on a timer to ensure the microorganisms have been exposed to a sufficient amount of light to kill or inactivate them or reduce them by a desired log reduction or a sensor may determine the amount of microorganisms and once the sensor has detected a reduction in microorganisms the lights are turned off.

Referring now to FIG. 6, the isometric view of another example embodiment of a UV light housing, shown as a drinking fountain push bar 62, and a drinking fountain guard 64. In one or more embodiments the UV light source 14 is a single light source in the push bar 62. However, the light source 14 may be multiple light sources, and could extend to also expose the sides of the drinking fountain to UV light rays. The light source 14 may be placed outside of the push bar 62, or a combination of internal and external placement. The push bar 62 can be made from any durable material such as metal, plastic, glass, fiberglass, and the like. The UV light sources 14 may be on a timer 15 to ensure the microorganisms have been exposed to a sufficient amount of light to kill or inactivate them. The timer 15 may be set to reduce the quantity of microbes on the contact surface 16 by a desired log reduction. For example, the timer may be calibrated to provide a 6-log reduction, or, in other words, be 99.9999% effective. The power source 12 may be a battery, a rechargeable battery, kinetic energy, photovoltaic array, power from the power grid or from the gas pump, or the like. The power source 12 may be internal, such as with a battery, or it may be external, such as with the power grid.

In one or more embodiments the guard 64 has a single light source 14 placed on at the external top of the guard. However, the light source 14 may be multiple light sources which may be placed internal, external, or a combination of internal and external placement on the guard 64. The light source 14 may be configured to expose the bubbler 68 to the UV light rays.

In one or more embodiments the UV light source 14 may be configured to be always on, or it may be configured to turn on once the public contact surface has been used, such as, for example, when a proximity sensor 66 senses a user approaching or leaving the area. The UV light source 14 may be on a timer to ensure the microorganisms have been exposed to a sufficient amount of light to kill or inactivate them or reduce them by a desired log reduction.

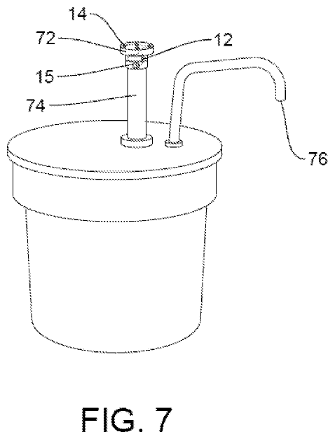

Referring now to FIG. 7, the isometric view of another example embodiment of a UV light housing, shown as a condiment dispenser with a pump handle 72. In one or more embodiments the UV light source 14 is a single light source in the pump handle 72. However, the light source 14 may be multiple light sources, and could extend to also expose the shaft 74 of the pump to UV light rays. The light source 14 may be placed outside of the pump handle 74, or a combination of internal and external placement. The pump handle 72 can be made from any durable material such as metal, plastic, glass, fiberglass, and the like.

In one or more embodiments the UV light source 14 may be configured to be always on, or it may be configured to turn on once the public contact surface has been used, such as, for example, when the shaft 74 is pushed down. The UV light source 14 may be on a timer to ensure the microorganisms have been exposed to a sufficient amount of light to kill or inactivate them or reduce them by a desired log reduction.

In some embodiments the UV light source 14 is integral to the high contact item, i.e., the door handle, gas pump handle, pen, drinking fountain button or condiment pump. In other embodiments, the housing 10 may be configured to be coupled to the high contact item and encompass the high contact item. The high contact item is covered with the housing 10 such that a user would now contact the housing when operating the high contact item, which kills or inactivates microbes as set forth above.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only and is not intended to limit the scope of the present disclosure, which is defined solely by the claims. Accordingly, embodiments of the present disclosure are not limited to those precisely as shown and described.

Certain embodiments are described herein, including the best mode known to the inventors for carrying out the methods and devices described herein. Of course, variations on these described embodiments will become apparent to those of ordinary skill in the art upon reading the foregoing description. Accordingly, this disclosure includes all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

What is claimed is:

1. A UV light system for a contact item, the system comprising:
a power source;
a transparent housing defining a system contact surface having a contact side and an inner side;
a plurality of UV light sources positioned on the inner side of the housing, wherein the plurality of UV sources are configured to illuminate the contact surface with UV light rays sufficient to provide at least a 2-log reduction in the number of microorganisms on the contact surface.

2. The housing of claim 1 wherein the power source is a rechargeable battery.

3. The housing of claim 1 wherein the power source is kinetic energy from the door opening and closing.

4. The housing of claim 1 wherein the lights are connected to a power grid

5. The housing of claim 1 wherein the power source is at least one photovoltaic panel.

6. The housing of claim 1 wherein the contact surface is found on a door handle, a door push plate, a pen, a drinking fountain guard, a drinking fountain push bar, or a condiment pump handle.

7. The housing of claim 1, wherein the housing conforms to an existing contact surface of a contact item such that the system contact surface replaces the existing contact surface.

8. A UV light system for a contact item, the system comprising:
a power source;
a transparent housing defining a system contact surface having a contact side and an inner side, wherein the housing is comprised of a first section and a second section detachably coupled to the first section and wherein the housing conforms to an existing contact surface of a contact item such that the system contact surface replaces the existing contact surface;
a plurality of UV light sources positioned on the inner side of the housing, wherein the plurality of UV sources are configured to illuminate the contact surface with UV light rays sufficient to provide at least a 2-log reduction in the number of microorganisms on the contact surface.

9. The system of claim 8, wherein the first section and second section are mirror images of each other.

10. A method of providing a disinfected contact surface of a contact item, the method comprising the acts of:
forming a housing to conform to an existing contact surface, wherein the housing is substantially transparent and defines a system contact surface having a contact side and an inner side, wherein the housing is comprised of a first section and a second section detachably coupled to the first section such that the system contact surface replaces the existing contact surface, wherein the housing further comprises a plurality of UV light sources positioned on the inner side of the housing and are configured to illuminate the contact surface with UV light rays;
connecting the first section and the section to replace the existing contact surface;
operating the UV light sources to illuminate the system contact surface sufficient to provide at least a 2-log reduction in the number of microorganisms on the system contact surface.

11. The method of claim 10, wherein the existing contact surface is replaced by the system contact surface by removing the existing contact surface and inserting the system contact surface.

12. The method of claim 10, wherein the existing contact surface is replaced by the system contact by attaching the first section to the second section about the existing contact surface.
